(19) United States
(12) Patent Application Publication
(10) Pub. No.: US 2015/0359886 A1
(43) Pub. Date: Dec. 17, 2015

(54) SYSTEM AND METHODS FOR NANOSTRUCTURE PROTECTED DELIVERY OF TREATMENT AGENT AND SELECTIVE RELEASE THEREOF

(71) Applicant: BROWN UNIVERSITY, Providence, RI (US)

(72) Inventors: Chia-Hsuan Wu, Campbell, CA (US); Jin Ho Kim, Providence, RI (US); Jingming Xu, Providence, RI (US)

(21) Appl. No.: 14/600,188

(22) Filed: Jan. 20, 2015

Related U.S. Application Data
(63) Continuation of application No. PCT/US2013/051482, filed on Jul. 22, 2013.
(60) Provisional application No. 61/673,784, filed on Jul. 20, 2012.

(51) Int. Cl. A61K 41/00; A61K 9/06; A61M 37/00; A61K 47/48; A61K 31/337; A61K 47/02; A61K 9/00

(52) U.S. Cl. CPC A61K 41/0052; A61K 47/02; A61K 9/06; A61K 9/0092; A61K 47/48215; A61K 31/337; A61M 37/00; A61M 2037/0007

(57) ABSTRACT

A method and nanoparticle construct provides shielded delivery of a drug or agent to a tissue or treatment site, and release of the agent may be triggered externally. Carbon nanotubes (CNTs) are filled with the therapeutic agent in a temperature sensitive gel, and release of the agent is effected by inductive heating, e.g. applying an alternating or pulsed magnetic field, or electrical field. The CNTs may be functionalized for solubility, drug absorption, responsivity to pH, enzyme catalysis, and/or ambient biological environment. Encapsulation within the nanostructure protects the intracorporal or surrounding cellular environment from the potentially toxic cargo and prevents the degradation of the cargo during delivery. By releasing at or in the target tissue extremely small amounts of the agent may achieve an effective level of treatment, as measured by cell apoptosis, tumor shrinkage or other treatment effect while safely avoiding systemic damage.

### RELATED APPLICATIONS

This international application claims the benefit of U.S. provisional application Ser. No. 61/673,784 filed Jul. 20, 2012 entitled, “System and Methods for Delivery of Nanostructure Treatment Agent and Temporally Regulated Release of Agent” by Chia-Hsuan Wu, Jin Ho Kim, and Jingming Xu, which is incorporated by reference herein in its entirety.

### GOVERNMENT FUNDING

A portion of this work was supported by a Multidisciplinary University Research Initiative grant sponsored by the following Department of Defense research offices: the Office of Naval Research, the Army Research Office and the Air Force Office of Scientific Research. The government has certain rights in this invention.

### TECHNICAL FIELD

The invention provides a system for delivery of a drug or other treatment agent to a target tissue or site and initiation of effective and temporally-regulated release of the agent at the tissue or site.

### BACKGROUND

Selective delivery of a therapeutic agent to a target site in the body which is released in a controlled manner can be an effective method of therapeutic agent delivery. Through scientific advancements in nanotechnology, functionalized nanomaterials are able to deliver therapeutic agents to target cells with enhanced drug absorption, and respond to changes in pH, enzyme catalysis, and temperature in the biological environment of the target site. Binder, Angewandte Chemie International Edition 47:3092-3096, 2008; Kam et al., Journal of the American Chemical Society 127:12492-12493, 2005; Keuretjes et al., Angewandte Chemie International Edition 48:9867-9870, 2009.

In cancer therapy, electromagnetic fields have been used for induction heating of magnetic nanoparticles, or for generation of mechanical forces via magnetic micro disks for physical destruction of cancer cells. Thomas et al., Journal of the American Chemical Society 132:10623-10625, 2010; Kim et al., Nature Materials 9:165-171, 2010. This method involved carrying the drug on the outside of the nanostructures, which results in some loss and degradation of the drug and poses a risk of the drug damaging surrounding biological environments during transport.

Hollow nanostructures as drug delivery carriers have many advantageous physicochemical properties. Carbon nanotubes (CNTs) are easily internalized by a cell; they have excellent biocompatibility, high surface area, high aspect ratio, and may exhibit metallic or semi-metallic behavior. Mintmire et al., Physical Review Letters, 68:631-634, 1992. Additionally, the outer surface of the CNTs can be chemically functionalized, and drugs can be carried within the inner cavity of the nanotubes. Kostarelos et al., Nature Nanotech, 4:627-633, 2009. Molecules of normally solid compositions loaded into the inner cavity of the CNTs, such as fullerenes, metal halides and small molecules, may require high temperatures for molten-phase loading. Hong et al., Nature Materials, 9:485-490, 2010. In addition, compositions of an agent selected for loading must have low surface tension if the composition is to be drawn into the CNTs by capillary force, van der Waals interactions or hydrophobic force. However the strong interactions between the inner surface of a CNT and the therapeutic agent make it difficult to release the agent from the CNT. Controlled loading and release of the therapeutic agent has therefore been considered problematic or even unattainable.

Development of an externally triggered drug release mechanism which exploits intrinsic properties of the CNT for effective drug release would therefore be highly desirable.

There is a need for a nanostructure drug carrier that is non-toxic to cells, and capable of high loading efficiency and highly effective releasing capacity.

### SUMMARY

This is achieved in accordance with the present invention by a method and associated construct for delivering a temporally regulated and biologically or spatially targeted therapeutic agent. The method includes: administering, in a delivery vehicle comprising a conductive nanostructure, a composition of the therapeutic agent and a temperature sensitive gel, wherein the composition is located and protected in an interior compartment of the nanostructure vehicle; providing the nanostructure vehicle with therapeutic agent to the target site; and applying induction heating to release the composition from the interior of the nanostructure at the target site.

Induction heating (also referred to as ‘inductive heating’) is the process of heating an electrically conducting object, such as a metal or metal-like material, by electromagnetic induction, i.e., generating Eddy currents (also known as Foucault currents) such that electron flow and intrinsic resistance results in joule heating of the object. In accordance with an aspect of the present invention, such heating induces a marked phase change in the temperature sensitive gel composition, initiating its release from the protected nanostructure containment.

Thus the nanostructure vehicle acts as a protective capsule for the therapeutic composition during parenteral or other administration, so that the therapeutic composition remains inaccessible to, and neither degrades nor is degraded by contact with, surrounding fluids or tissue, until its release at the target delivery site by inductive heating. Furthermore, the delivery vehicles may be very selectively targeted by coatings, by inclusion in a targeting emulsion formulation, or by suitable surface functionalization such that the delivery vehicles pass in the bloodstream without being scavenged by the liver or the immune system, and instead accumulate at a desired target tissue, such as a specific tumor or target organ tissue. In addition, the nanostructures may be of a size selected such the delivery vehicles are taken up by cells of the targeted tissue. That is, delivery results in the nanostructures adhering to the cell surface or entering the cell interior by endocytosis. Preferably the nanostructures are metal or have metal-like conductive characteristics (for example, the nanostructures are suitable gold or silver nanoparticles, or carbon nanotubes), and possess an interior that may be filled with the gel/agent composition for protection during delivery by the nanoparticle.

In an embodiment of the method and compositions of the invention, the nanostructures are nanotubes, such as carbon or metal nanotubes. In a carbon nanotube embodiment, the method may include, prior to administering the delivery vehicles, the step of aligning nanostructures in an array and loading the composition as a fluid into the aligned nanostructures by applying the composition to a first side of the array (to an open end such as the top end of the nanostructures); and, drawing the composition into the nanostructures by applying a suction or vacuum at a second side of the array (e.g., bottom ends of the nanostructures), thus drawing or loading the composition into the interiors of the nanostructure. The fluid may be a hydrogel in its liquid or sol state, and may contain a desired treatment agent as smaller nanoparticles or a nanoemulsion dispersed and suspended in the liquid hydrogel. Thus, the treatment agent need not be a soluble agent but may be insoluble, or a sparingly soluble, such as the chemotherapy agent Paclitaxel or Taxol, and/or the treatment potentiator C6-ceramide. In such cases, the treatment agent is dispersed in the gel in a suitable density or concentration prior to loading the gel/agent composition into the nanoparticles. In an aspect of the invention, an extremely toxic or potent chemotherapy agent may thus be encapsulated and isolated within the nanotubes during delivery, thus preventing adverse interactions with the body, and then released directly at the target site, such as a tumor, thereby providing concentrated and localized toxicity. The invention thus provides an effective means to deliver an effective treatment dose of a drug to a target tissue, while minimizing collateral or systemic exposure.

In an embodiment of this method, the nanostructure array is an array of carbon nanotubes that are formed by chemical vapor deposition (CVD) growing the nanostructures on an inner wall of pores in an ordered and uniform anodic aluminum oxide (AAO) nanopore array template. The nanotubes thus formed may be conveniently filled when still bound in an array, through one side of the template, and once filled, may be released by dissolving the template. In other embodiments, the nanostructures are gold nanotubes, gold nanocages or other suitable hollow, electron-conducting nanostructures.

In an embodiment of this method, applying induction heating to the target site further includes directing to the target site at least one energy source selected from the group of: alternating current (a.c.) magnetic field, pulsed magnetic field, and electrical field that is effective to heat the nanotubes; such heating causing the gel to undergo a phase-change transition and pass from the interior of the nanotubes as a liquid at the target site. In an embodiment of this method, the field strength, frequency of the a.c. magnetic field, and duration of the applied field used to cause induction heating are chosen to be effective to sufficiently heat the nanostructures' interior to bring about the phase change of the hydrogel composition for release of the composition from the nanostructure.

When used for medical diagnostic or treatment applications, such as chemotherapy, the hydrogel may be selected or compounded such that the phase change occurs at a temperature above body temperature to assure that the treatment agent remains encapsulated during the delivery process, but at a temperature sufficiently close to body temperature to assure that the inductively-produced heating suffices to achieve transition. This may be done by selecting a suitable pure or pharmaceutically acceptable hydrogel or gelatin formulation, or by mixing two or more different compatible gel materials, which may include common natural gelatin compositions as well as appropriately reactive synthetic oligomers or polymer precursors, such that the selected hydrogel or mixture of gels and/or precursor materials possesses an effective transition point at a temperature attainable by inductive heating of the nanostructure shell. The liquid hydrogel formulation may also or alternatively be selected or compounded such that the phase transition is characterized by a change in volume, hydrophilicity or other characteristic at the transition temperature which causes, or further promotes or amplifies the release of the treatment agent from the nanostructure at the phase transition point. For example, when the nanotube structure is inductively heated, a polymer that undergoes a large change in volume may be effectively expelled or ‘squirted out’ from the nanotubes, or may potentially be sufficiently energetic to rupture or open the tube wall and release the enclosed cargo to contiguous tissue. Such a volume-change transition characteristic is especially useful in view of the large magnitude of forces generally required to exit the small-dimensioned interior of the nanotube structure, and the disparate range of hydrophilic or hydrophobic properties of the nanotube wall and of the gel/agent contents.

The method may further include the step of observing intracellular uptake of the nanostructures into, or observing the presence of the nanotubes at, the targeted cells or tissue prior to applying the inductive heating field. For example, when the nanostructures are administered parenterally to an animal, or are presented in a culture medium, and either contained within or functionalized with a targeting structure that causes them to accumulate at and/or to be endoeytosed in a targeted tumor, tissue or organ, the step of observing may include imaging the target tissue or the affected cell culture to confirm such uptake. For this purpose, the delivery vehicle may include an appropriate image contrast enhancement agent, such as an MRI or X-ray imaging enhancer or a fluorescent marker; or detection of the nanoparticles in the body may be otherwise enhanced by special processing of the underlying imaging signals. When used in clinical tests and pharmacological development or assay trials, the method may further include monitoring, observing or measuring amounts of cell survival, or tumor size, prior to and after application of the alternating magnetic field to determine, confirm, quantify or adjust treatment dosage and efficacy as appropriate considering the treatment site, nanostructure delivery modality and target tissue characteristics.

In an embodiment of this method, the therapeutic agent is selected from at least one of a low molecular weight drug, an inorganic compound, and a biomolecule. In an embodiment of this method, the biomolecule is at least one of a plasmid, a peptide, a polysaccharide, a protein, an enzyme, a hormone, a neurotransmitter, a metabolite, a lipid, a sterol, a siRNA, or a virus or viral product, or a biomolecular adduct.

In an exemplary embodiment of this method, prior to loading the method further includes combining the therapeutic agent and the temperature sensitive gel at a temperature above the transition temperature of the temperature sensitive gel, such that the gel is a fluid. In an embodiment of this method, the nanostructure may include one or more selected from among a nanotube, a nanocone, a nanohorn, a nanoporous structure, and a nanocage. In an embodiment of this method, the nanostructure is made from a material of at least one of a carbon, a gold, a silver, a platinum, an iron, a cobalt, an iron-platinum, an iron-cobalt, a conductive polymer, a silicon, and a metal ferrite. The metal of the metal ferrite may be manganese, iron, cobalt, nickel or zinc.

In an embodiment of this method, the temperature sensitive gel includes at least one of an aqueous solvent and a non-aqueous solvent. In an embodiment of this method, the temperature sensitive aqueous gel comprises at least one of a gelatin, a starch, an agar, an agarose, a poly(ethylene oxide) (PEO), a poly(N-isoproprylacrylamide) (pNIPAAm), and a poly(propylene oxide) (PPO). Other suitable polymers with glass transition temperature between 32° C. and 45° C. may include polymers: poly(N,n-diethylacrylamide) (32° C.), poly(N-isopropylmethacrylamide) (44° C.), poly(N-cyclopropylacrylamide) (45.5° C.), hydroxypropyl cellulose (45° C.), methyl cellulose (40-50° C.), hydroxypropylmethyl cellulose and ethylhydroxyethyl cellulose.

In an embodiment of this method, the gelatin is made from materials of at least one of a bovine skin, a bovine bone, a bovine hide, a human bone, a porcine skin, a porcine bone, a cattle bone, a cattle skin or a fish part (e.g., fish skin). The gel or composition of gel materials, whether natural or synthetic, is suitably purified for medical use or use in tissue cultures. In an embodiment of this method, the non-aqueous solvent may be a hydrophilic solvent such as ethanol or other alcohol, or may be an ether or other solvent. The non-aqueous solvent may be a solvent for the therapeutic agent, which may, for example be insoluble or sparingly soluble in aqueous. In an embodiment of this method, the gel-sol transition temperature for the gelatin loaded into the nanostructures, the glass transition temperature of the polymer, or more generally the transition temperature for the hydrogel or polymer formulation loaded in the nanostructures is about between 32° C. and 45° C. Preferably the transition temperature when used for therapy in a mammal is above the normal body temperature of the mammal, for example, above 39-40° C. for a human.

An embodiment of the invention provides a nanoparticle preparation for targeted intracellular drug delivery and temporally regulated release including: a nanostructure, having open ends and a hollow nanotube configuration; and, a composition, including a therapeutic agent and a gel loaded in the hollow interior of the nanostructure. In an embodiment of this device, the hollow nanostructure is at least one of a nanotube and a nanocage. In an embodiment of this device, the hollow nanostructure is made from at least one material selected from a carbon, a gold, a silver, a platinum, an iron, a cobalt, an iron-platinum, an iron-cobalt, a conductive and a silicon.

In an embodiment of the nanoparticle preparation, the gel is characterized by a temperature-induced phase-change transition from a solid or gel to a sol or liquid as a function of temperature, such that the composition is released from the nanostructure for delivery. In an embodiment of the device, the temperature sensitive gel is at least one of a gelatin, a starch, an agar, an agarose, a poly(ethylene oxide) (PEO), a poly(N-isoproprylacrylamide) (pNIPAAm), a poly(propylene oxide) (PPO), poly(N,n-diethylacrylamide), poly(N-isopropylmethacrylamide), poly(N-cyclopropylacrylamide), hydroxypropyl cellulose, methyl cellulose, hydroxypropylmethyl cellulose and ethylhydroxyethyl cellulose. In an embodiment of the device, the gelatin is made from materials of at least one of a bovine skin, a bovine bone, a bovine hide, a porcine skin, a porcine bone, and a human bone.

In an embodiment of the nanoparticle preparation, the hollow nanostructure further includes an exterior surface that is chemically functionalized for at least one function selected from: solubility, drug absorption, responsivity to pH, responsivity to enzyme catalysis, and responsivity to ambient biological environment temperature. In an embodiment of the device, the exterior nanostructure surface is treated with nitric acid, such that functionalized cellular uptake of a resulting nitric acid-treated nanostructure is greater than that of a control hollow nanostructure not so treated and otherwise identical. In an embodiment of the device, the exterior nanostructure surface is non-covalently functionalized with phospholipid-polyethylene glycol (PL-PEG) such that a resulting PEG-functionalized nanostructure is more hydrophilic and displays prolonged circulation in the bloodstream in comparison to a control nanostructure not so functionalized. In an embodiment of the device, the PEG-functionalized nanostructure comprises nanotubes of about 50 nm diameter and about 200-1000 nm length. The diameter is determined by initial nanotube fabrication conditions, e.g., the fabrication of a porous anodic aluminum oxide (AAO) template, and is relatively large so as to facilitate loading and enable loading of a therapeutically sufficient amount of the treatment agent therein, while the nanotube length may be determined by a post-loading processing step, dissolving the templates and shortening the freed nanotubes.

In an embodiment of the nanoparticle preparation, the nanostructure is functionalized to further include amine-terminated polyethylene glycol phospholipids (PL-PEG-NH₂) and has a plurality of amine terminals for further conjugation of biomolecular adducts. In addition to stable PL-PEG preparation, polyvinyl-pyrrolidone (PVP) or polystyrene sulfonate (PSS) may be used to prepare solutions for carrying a high weight fraction of loaded nanotubes, and/or the nanotubes may be dispersed in organic solvents such as DMF, DMAc (dimethyl acetamide) or DMP (dimethyl pyrrolidone). A number of surfactants may also prepare the nanotubes for aqueous dispersion, such as amylose (a glucose-based natural polymer), sodium dodecyl sulfate (SDS), dodecyl-benzene sodium sulfonate (NaDDBS), cetyltrimethylammonium bromide (CTAB) and Oπ-10 (polyethylene oxide (10) nonylphenyl ether). Non-covalent surfactant or polymer treatments allow one to adsorb various groups on the nanotube surface without disturbing the sheet- or wall-nanostructure of the nanotubes.

Reference is further made to jointly-authored article entitled “Trojan-Horse Nanotube On-Command Intracellular Drug Delivery” by Chia-Hsuan Wu, Cong Cao, Jin Ho Kim, Chih-Hsun Hsu, Wayne D. Bowen Harold J. Wanebo, Jimmy Xu and John Marshall. Nano Lett. 2012, 12: 5475-5480. That article is hereby incorporated herein by reference in its entirety for its detailed technical discussion of a proof-of-principle example.

### BRIEF DESCRIPTION OF DRAWINGS

These and other features and aspects of the invention will be understood from the examples below taken together with illustrative drawings, wherein

FIG. 1A schematically shows loading into a carbon nanotube (CNT) and the temporally regulated release from CNTs of a composition, which is comprised of a hydrogel polymer and a therapeutic agent. The composition is loaded into the CNT by vacuum suction and the composition is released from the CNT by applying inductive heating to the CNT.

FIG. 1B is a set of transmission electron microscopy (TEM) images showing the successful loading of Quantum Dots (QDs) nanocrystals into a CNT that has a diameter of 50 nm.

FIG. 1C is a set of images taken under an epifluorescence microscope, with and without inductive heating. The first image shows that no green luminescence light was emitted from the QDs when the alternating current (a.c.) magnetic field was turned off. The second image shows green luminescence light was emitted from the QDs when the alternating current magnetic field was turned on. The images show that the CNT therapeutic agent delivery method has the ability to temporally regulate the release of a CNT loaded therapeutic agent.

FIGS. 2A-2D show bar graphs of short-chain C6 sensitization and Taxol-induced cell death of three different pancreatic cancer cell lines (A, B: L3.6; C: PANC-1; D: MIA PaCa-2) in experiments to determine sensitivity to a treatment agent; a relatively high concentration of C6-ceramide (5-10 micrograms per milliliter) is needed to reach the chemosensitization effect.

FIG. 3A is an image of fluorescently labeled pancreatic cancer cells taken under confocal fluorescence microscopy, showing internalization of CNTs in the cancer cells.

FIGS. 3B and 3C are vertical (3B) and horizontal (3C) cross sectional image views of fluorescently labeled cancer cells taken under confocal fluorescence microscopy. The images show that the CNTs were successfully internalized inside the pancreatic cancer cells.

FIG. 3D is a set of images showing a cell survival study by an in vitro cytotoxicity assay of pancreatic cancer cells treated under different conditions. There were three groups of pancreatic cancer cells: the first was a control group, the second was a group internalized with CNTs, and the third was a group internalized with CNTs containing the chemotherapeutic drug Taxol. All groups were exposed to an alternating magnetic field that was turned either ‘on’ for 30 minutes or ‘off’. The FIGURE shows that the application of an a.c. magnetic field for 30 minutes to pancreatic cancer cells internalized with CNTs containing Taxol, resulted in 70% cell death with release of Taxol from the CNTs.

FIG. 3E is a bar graph showing the percent cell survival of pancreatic cancer cells at the same conditions as described in FIG. 3D.

FIGS. 3F and 3G illustrate results of a histone-DNA ELISA assay (3F), and several biochemical surrogate markers (3G) to confirm the pattern of apoptosis for the CNT-Taxol-C6 treatment.

### DETAILED DESCRIPTION

The present invention involves a nanoparticle-based method for the controlled delivery of a cargo, such as a toxic chemotherapy agent or other treatment agent, or an imaging or diagnostic agent, to a target tissue site in a body while isolating and protecting the cargo against release, degradation by, or interaction with the body during passage through the body to the site, and permits release of the cargo at the site. An exemplary embodiment is described herein based on carbon nanotubes (CNTs) filled with the cargo. The hollow nanostructure encapsulates toxic cancer drugs and/or drug potentiators in a matrix of a temperature sensitive gel such that the cargo is effectively sealed within and isolated by the surrounding tube structure, but may be released by inductive heating, which is induced by applying an alternating or pulsed magnetic field.

In the examples discussed herein, suitable CNTs are grown on the inner walls of a highly ordered and uniform anodic aluminum oxide (AAO) nanopore array template by chemical vapor deposition (CVD); the nanotube walls are electrically conductive, and the tube interior dimensions are of a size to carry and release an effective treatment amount of the cargo/drug to the target cells or tissue, and also, in some embodiments, of a size such that the CNTs are taken up or endocytosed by cells of the targeted tissue or tissue culture.

For general processing steps and treatment methodologies governing CNT manufacture and the control of dimensions, wall thickness and electrical characteristics, and for filling procedures for various media, inclusions and agents, reference is made to the survey article Carbon nanotubes prepared by anodic aluminum oxide template method. HOU P. X et al. Chin Sci Bull 2012, 57:187-204. Dimensions such as tube diameter, length and wall thickness are readily controlled, as well as the processes for deposition of carbon so as to deposit single-walled or multi-walled tubes, and so as to control electrical conductivity of the resulting wall material (such as carbon tubes formed with multiple layers of graphene). In general, for inductive heating of the nanotubes, the axial, radial and circumferential Joule heating power P (W/kg) of Eddy current heating will all follow the form

P∝B²ω²(Rout²−Rin²)L/ρ

where B is peak flux density magnetic field (T), L is the length of the nanotube (m), ω is the frequency (Hz), ρ denotes the resistivity of the nanotube (Ωm), and Rout and Rin are the outer and inner radii of nanotube, respectively. Thus, the extent of heating generated in each nanotube is controlled by the applied magnetic field strength, frequency, and by the tube's conductivity and physical dimensions, the former two parameters being field and application adjustable, whereas the latter two are controllable by the tube fabrication process and are readily addressed in the AAO template platform.

Carbon nanotubes (CNTs) as drug delivery carriers have many exceptional physiochemical properties. They are biocompatible hollow structures with a large surface area, high aspect ratio, and have metallic or semi-metallic behaviour. The outer surface of the CNT can be chemically modified, while the inside cavity of the nanotubes can be accessed with drugs. Metal halides and small molecules have previously been encapsulated in carbon nanotubes. However, the encapsulation has generally involved high-temperature molten-phase loading (e.g. 900° C.), or else the selection of cargo was limited to molecules with low surface tension so that they can be drawn into the nanotubes by capillary force or van der Waals forces, making it inherently difficult to release the agents controllably from the CNTs. In addition to the access problem, there are several challenges to engineering a nanotube drug carrier with: 1) high loading efficiency; 2) high drug retention rate over the delivery process; and 3) “on-command” release. This work presents a new strategy that solves these impeding factors to controlled loading of the therapeutic agents and deploys an a.c. magnetic field as an external command for controlled drug release.

In contrast to conventional CNTs which have an interior space that is small and difficult to access, the CNTs used here have a relatively large diameter (˜40 nm inner diameter). They are fabricated in an array form with a perfectly aligned vertical orientation by growing the CNTs in a highly ordered and uniform anodic aluminum oxide (AAO) nanopore array template by chemical vapour deposition (CVD), following which they undergo mechanical or chemical treatment to open both ends. The opening of both ends of the nanotubes while embedded in the template provides access to the interior space for bulk filling with the cargo.

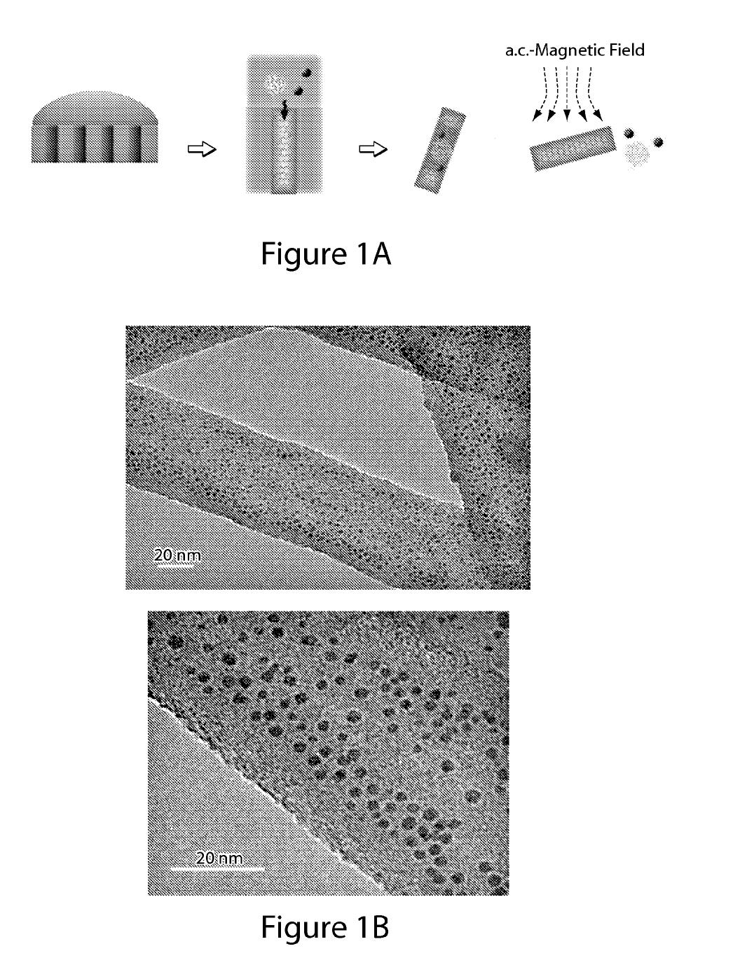

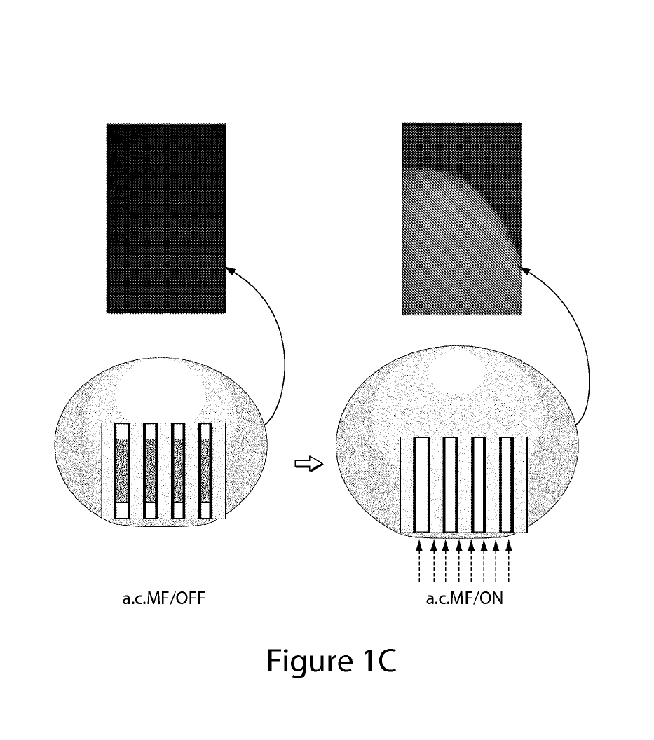

Drug loading was accomplished by depositing droplets of the drug solution on top of the vertically aligned nanotube array membrane while applying vacuum suction through a filter at the bottom (FIG. 1A). After loading, the individual CNTs were released by dissolving away the alumina template; the chemical inertness of the CNT tube walls protects their contents from solvation or damage.

Initially, to assess the loading and encapsulation efficiency, an aqueous solution of Quantum Dots (QD, CdSe/ZnS nanocrystals) was loaded into the CNT array. Encapsulation was verified under transmission electron microscopy (TEM). As shown in FIG. 1B, TEM images of QD-loaded CNTs demonstrate the successful encapsulation, and the QDs can clearly be seen to have been densely loaded in the interior of the nanotube.

The AAO template-synthesized carbon nanotubes are intrinsically conductive, yet tend to possess higher electrical resistivity, than carbon nanotubes synthesized in other processes, such as by arc-discharging. This higher resistivity is beneficial for purposes of the present invention because the CNTs have sufficient electrical resistivity to generate heating via magnetic field induction. As noted above, the heating generated in each nanotube is controlled by the applied magnetic field strength, frequency, and by the conductivity and physical dimensions of the tube. The latter two parameters are addressed in the AAO template-based fabrication process.

By incorporating a temperature sensitive hydrogel mixed with the drugs of interest as the cargo, then selectively applying inductive heating to cause the hydrogel to undergo a gel-sol phase transformation, the drug payload is controllably released of out of the CNTs. In the absence of inductive heating, the surface tension and viscosity keep the gel-drug payload inside the tube. Furthermore, the surface tension of water prevents external water from entering the nanotube to displace the drug payload. Feasibility, safety and efficacy of the invention were experimentally confirmed as reported below.

### Methods and Materials

Taxol/C6-Ceramide Loading into CNT Arrays.

The CNT array is fabricated on anodized aluminum oxide (AAO) template using a CVD process and opened on both ends by chemical treatments¹⁴. The drug loading in our case was by applying the mixture of Taxol/C6-ceramide and gelatin (0.2 g/mL, Sigma-Aldrich) solution on top of vertical aligned nanotube array and vacuum suction at the bottom. After loading, the individual CNTs were released from alumina template by 0.1 M NaOH. The solution was then filtered through filter paper (Millipore, pore size 50 nm) to remove excess NaOH, and washed thoroughly with deionized water. The released CNTs were first treated with diluted nitric acid to reduce their length to 200-1000 nm. Afterwards, CNTs were further sonicated with amine-terminated phospholipid-polyethylene glycol [PL-PEG, 0.1-1 mg/mL of DSPE-PEG (2000) Amine, Avanti Polar Lipids Inc.] for 1 h. The mixture was then filtered through filter paper and washed thoroughly with water or buffer.

### Antibodies and Reagents.

C6-ceramide was obtained from Avanti Polar Lipids Inc. Taxol was supplied by RI Landmark Medical Center. p-Akt(Ser 473), Akt1/2 and cleaved-caspase 3 primary antibodies were obtained from Cell Signaling Tech. Mouse mono-clonal antibody against β-actin was obtained from Sigma.

### Cell Apoptosis Assay (Histone DNA-ELISA).

The Cell Apoptosis ELISA Detection Kit (Roche, Palo Alto, Calif.) was used to detect pancreatic cancer cell apoptosis after indicated treatments according to the manufacturer's protocol. Briefly, the cytoplasmic histone/DNA fragments from cells with treatments were extracted and bound to immobilized anti-histone antibody. Subsequently, the peroxidase-conjugated anti-DNA antibody was then added for the detection of immobilized histone/DNA fragments. After addition of substrate for peroxidase, the spectrophotometric absorbance of the samples was determined by using a Perkin Elmer 1420 multilable counter at 405 nM.

### Cell Culture, Viability Assay and Western Blots.

Human pancreatic cancer lines PANC-1, MIA PaCa-2 (MIA) and L3.6 cells culture, cell viability assay (MTT dye assay) and Western blots assay were performed as described in Cao, C., et al. Galpha(i1) and Galpha(i3) are required for epidermal growth factor-mediated activation of the Akt-mTORC1 pathway. Science signaling 2, ra17 (2009).

FIG. 1A schematically illustrates the method of the present invention which involves the steps of filling the interior of nanotubes with a cargo, delivery of the filled nanotubes (e.g., via the bloodstream), and release of the cargo by application of an alternating magnetic field to inductively heat the nanotubes. Feasibility of loading was initially confirmed by loading a gel containing quantum dots (QDs) into the CNTs and imaging the loaded tubes. QDs with an approximate diameter of 4 nm were used. FIG. 1B, left panel, is a TEM image showing the high loading yield of QDs into nanotubes achieved. FIG. 1B, right panel is a magnified view of the TEM micrograph showing the QDs distributed inside a nanotube.

The efficacy of CNT loading in isolating the drug cargo from the surrounding environment and then releasing the cargo was first tested by imaging the release of a gel-QD loaded cargo from nanotubes in water when subjected to a 25 kHz a.c. magnetic field. The panels of FIG. 1C demonstrated that cargo release, as evidenced by green luminescence, was only observed in solution after the a.c. magnetic field was applied, indicative of QD release by inductive heating.

With the success of on-command QD release by inductive heating thus confirmed, the Trojan-Horse nanotube system was then tested for the delivery of non water soluble chemotherapeutic drugs that are cytotoxic to non-cancer cells at the therapeutic dose. We chose an in vitro cell model of pancreatic cancer, an aggressive malignancy with a five-year mortality over 95%, to test CNT based on-command chemotherapeutic drugs release and following anticancer effect. Paclitaxel (Taxol), an anti-microtubule agent, is a commonly used anticancer drug, but its application has been limited due to water insolubility and side-effects such as hypersensitivity reactions and the acquisition of chemo-resistance. Similarly, although recent studies suggest that C6-ceramide can dramatically increase the efficacy of chemotherapeutic agents including Taxol, doxorubicin and histone deacetylase inhibitors (HDACi), the systematic use of C6-ceramide is limited because of its insolubility, and will require the development of carrier based delivery methods. The CNT delivery-release system of this invention, offering isolation during delivery and release at the target site offers the possibility of improved administration for such drug combinations.

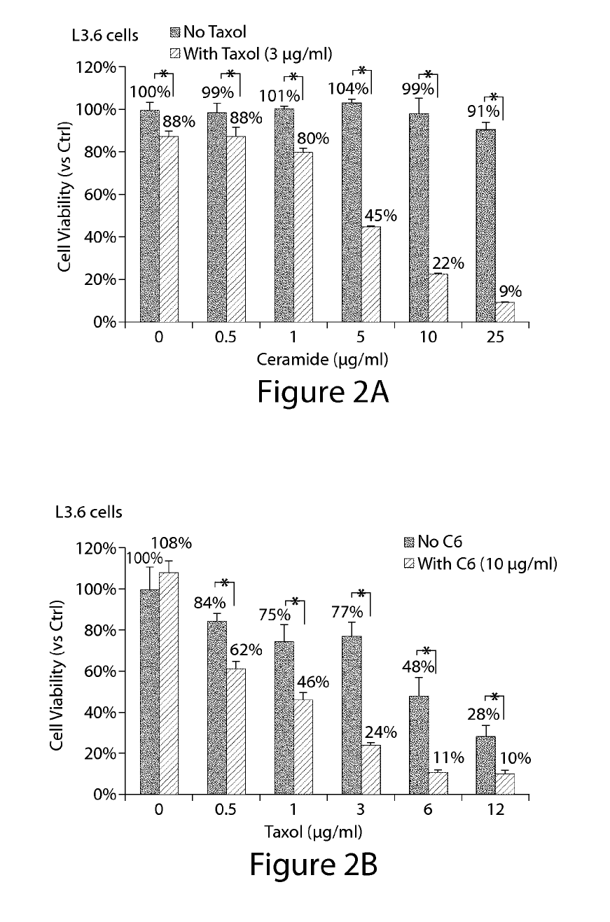

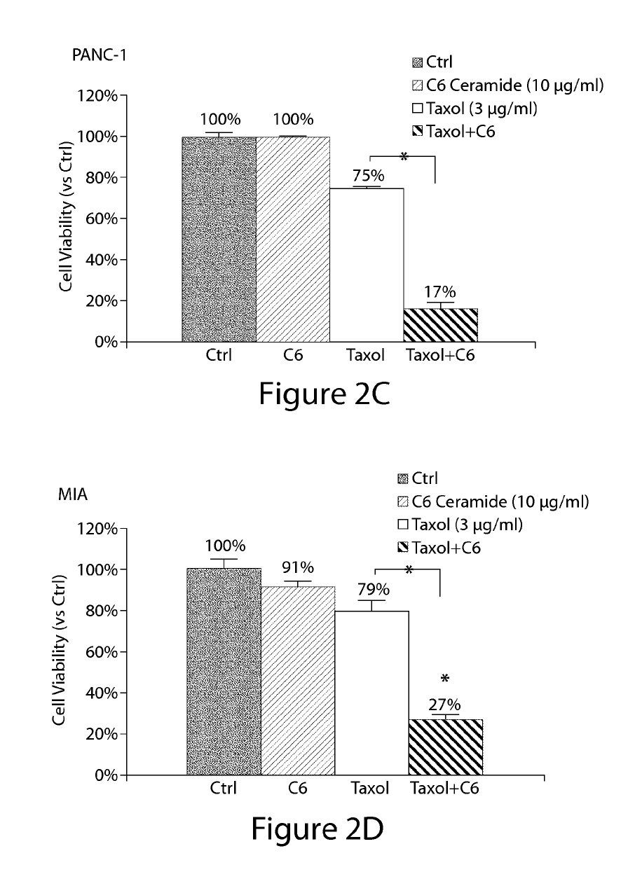

Accordingly, experiments were carried out to examine the magnitude of chemosensitization of C6-ceramide on Taxol in different cancer cell lines. The pancreatic cancer cell lines L3.6. PANC-1 and MIA PaCa-2 (MIA) cells were selected for this experiment. Results are shown in FIGS. 2A-2D. Even at high doses, Taxol alone or C6-ceramide alone had a limited killing of the three pancreatic cancer cells lines, whereas the combination of these two agents at a relatively low dose potently produced cell death (FIG. 2). These results suggest that C6-ceramide dramatically sensitizes Taxol-induced pancreatic cell death and therefore could be a useful adjuvant. However, the side effects noted above remain a challenging problem in this combination regimen and calls for a new delivery method.

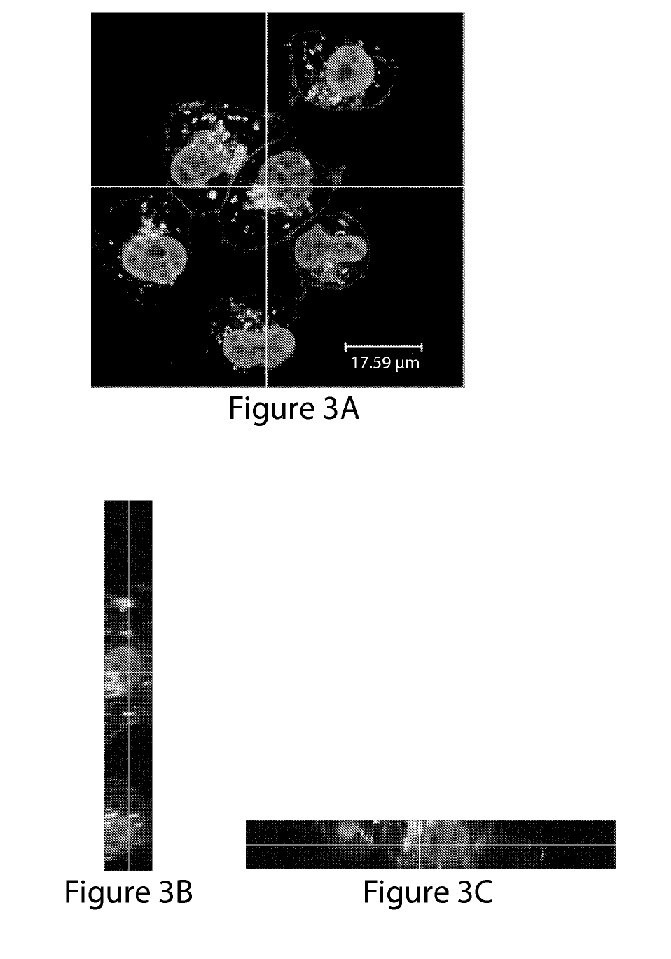

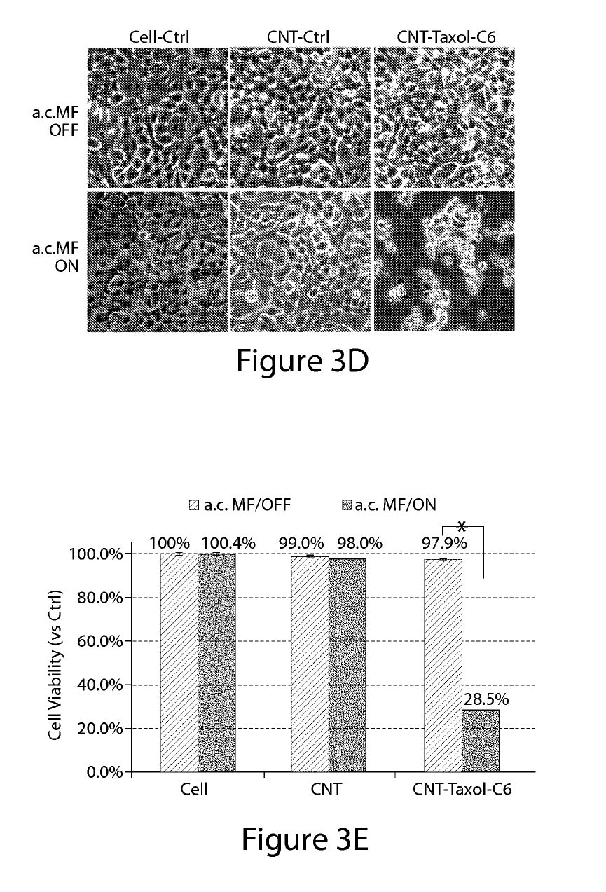

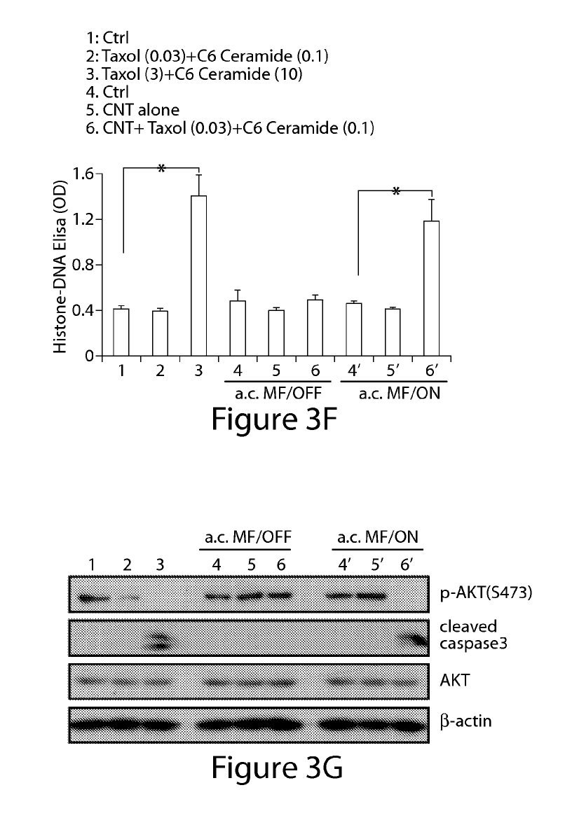

To verify that CNTs can be taken up by pancreatic cancer cells, the CNTs were first labelled the CNTs with Texas Red by conjugating Texas Red-X succinimidyl ester (Invitrogen) to CNTs that were non-covalently functionalized with amine-terminated polyethylene glycol phospholipids (PL-PEG-NH₂). The fluorescence labelled CNTs were added to the L3.6 cells overnight, then Texas Red in the cells was observed under confocal fluorescence microscopy. From the planar view of cells (FIG. 3A) and the cross-sectional view of cells (FIGS. 3B and 3C), Texas Red was seen on inside the pancreatic cancer cells, showing that CNTs were in fact taken up by these cells.

Using same pancreatic cancer cells (L3.6), the inductive heating release was we next tested the on-commandinductive-heating release of the encapsulated chemotherapeutic agents (Taxol and C6-ceramide) from CNTs. The drug-loaded CNTs were added to L3.6 cells for 12 hours, allowing them to be taken up by the cells. The cells were then washed with fresh basal medium (DMEM) 5 times to remove residual CNTs from the media, and a 30 min a.c. magnetic field (25 kHz) was applied to these cells to release the encapsulated drugs (Taxol and C6-ceramide) present in CNTs within the cells. After 48 h, the release of the drugs was inferred and assessed by the degree of cell death. We found that this process resulted in 71.5% of cell death (P<0.05 vs Ctrl) as reflected by cell viability loss seen in the MTT dye assay results of FIG. 3E and cell morphology change (shown in the lower right panel of FIG. 3D). When the alternating magnetic field was not applied, the Taxol/C6-ceramide loaded CNT were harmless to the cancer cells, with a 97.9% viability (p>0.05 vs Ctrl), indicating that the CNTs effectively isolated the toxic drug cargo from the surroundings, and that the a.c. magnetic field is required for the “on demand” release of drugs encapsulated in the CNTs. Importantly, no noticeable drop in cell viability was observed in cells incubated with the empty non-drug loaded-CNTs (FIGS. 3D and 3E, middle panels), indicating that both the empty CNTs and the a.c. magnetic field used here are safe to cells, with a 98-99% viability (P>0.05 vs Ctrl).

The cancer cell deaths caused by release of the enclosed/encapsulated drugs were further associated with cell apoptosis (FIG. 3F) and Akt inhibition (FIG. 3G), which is similar to the cell fate when Taxol/C6-ceramide was exogenously added directly. As shown in these Figures, only group #6′ [CNT+Taxol (0.03 μg/ml)+C6-Ceramide(0.1 μg/ml)] with on-command a.c. magnetic field ON had increased Histone-DNA ELISA OD, an indicator of cell apoptosis (FIG. 3F). Importantly, the drug concentration loaded into Trojan-Horse CNTs was 100 times lower (Taxol: 0.03 μg/ml/C6-ceramide: 0.1 μg/ml) than the concentration required for a comparable effect with the exogenously applied treatment (Taxol: 3 μg/ml/C6-ceramide: 10 μg/ml). The same low concentration of Taxol+C6-ceramide (Taxol: 0.03 μg/ml/C6-ceramide: 0.1 μg/ml) used in CNTs failed to cause cancer cell death when added exogenously (No CNT carrier, group 2^{#}). The 100 fold difference between these two groups indicates that Trojan-Horse CNTs effectively transport these drugs into the cells and can “on-demand” release the drugs to cause cell death. Hence, Trojan-Horse CNTs as drug delivery vehicle has the potential benefit of greatly reducing drug side effects.

### Discussion

Overall, the use of Trojan-Horse CNTs as a therapeutic drug delivery carrier has a number of advantages over traditional methods of chemotherapy delivery and treatment. The unique structure and the selected dimensions of these CNTs make them easy to fill with drugs, and once the drugs are loaded, internal storage is assured inside the CNTs until release by inductive heating. The surrounding gel medium may be compounded to enhance the storage life, and the encapsulation process itself protects the cargo from premature or unwanted interactions during delivery into the body. The external nanotube surface may be functionalized for targeted delivery to a specific tumor or molecular target or cell surface characteristic, such that the cargo is ‘delivered’ before the release is initiated. The amount of the inductive heating required for release of encapsulated drugs—a few degrees, localized at the nanotube having small total mass or volume—was found to have no detectable deleterious effect on the cells. Finally, while it is known, for example, that the combination of Taxol with C6-ceramide synergistically kills cancer cells but suffers from side effects; the efficient delivery of these drugs using CNTs allowed the required concentration of the drugs to be reduced 100 fold and safety contain the drugs until the release command. Thus, the experimental protocol verified a great reduction in collateral or systemic toxicity achievable by CNT encapsulation and delivery.

The foregoing description has focused on the delivery of chemotherapeutic drugs. However, the same Trojan-Horse CNT delivery system is applicable to deliver other cargo, such as plasmids, siRNA, growth factors, viruses and even metallic and atomic substances. Moreover, the delivered cargo need not be directed solely to destruction of tumors, tissues or cells, but may be a cargo used to effect genetic remediation, to program or influence the development of particular cells, or to alleviate or cure deficiencies or diseases.

The examples and experiments described above confirm the efficacy of the invention in isolating a toxic or therapeutic cargo and releasing or expelling the cargo on-demand at or within targeted tissue cells, thereby allowing order-of-magnitude or greater reductions in the magnitude of an effective treatment dose. As such, the methods and the nanotube preparations of the invention are expected to greatly advance the possibilities for treatment of cancers, while greatly reducing the systemic toxicity and adverse effects associated with chemotherapy and other treatments employed in cancer treatment. Extremely toxic drugs can be effectively isolated in nanoparticles from surrounding tissue or fluids, and the loaded nanoparticles themselves are safe and non-toxic until controlled inductive heating causes release of their cargo at or within target cells, thus providing a new and effective mechanism for highly efficient chemotherapy drug delivery at substantially lower doses and/or with immiscible, insoluble and/or highly toxic agents directly to targets.

1. A method for delivering therapeutic agent to a tissue site, the method comprising the steps of
administering the therapeutic agent to the tissue site in a delivery vehicle comprising a conductive nanostructure that contains or encloses a composition comprising the therapeutic agent in a hydrogel or temperature-sensitive gel, and
applying an electromagnetic field to the target site to inductively heat the conductive nanostructure and thereby release the agent and hydrogel at the target site.

2. The method according to claim 1, wherein, the conductive nanostructure includes carbon nanotubes (CNTs) and the method further comprises the step of loading the composition into the CNTs while CNTs are aligned in an array by
applying the composition in liquid formulation to a first side of the array, and
applying suction to a second side of the array to draw the composition into the CNTs.

3. The method according to claim 2, wherein the therapeutic agent is selected from at least one of a low molecular weight drug, an inorganic compound, and a biomolecule such as a biomolecule which is at least one of a plasmid, a peptide, a polysaccharide, a protein, an enzyme, a hormone, a neurotransmitter, a metabolite, a lipid, a sterol, a siRNA, and a biomolecular adduct.

4. The method according to claim 2, the step of aligning the structures in an array is performed by growing the nanotubes on an inner wall of an ordered and uniform anodic aluminum oxide (AAO) nanopore array template by chemical vapor deposition (CVD).

5. The method according to claim 1, wherein, the step of inductively heating is performed to elevate temperature of the composition in the conductive nanostructures to a gel-sol transition temperature or phase transition that undergoes a change of volume, hydrophilicity or other characteristic to release the agent and hydrogel from the interior of the nanostructures.

6. The method according to claim 1, wherein, the therapeutic agent is present as nanoparticles suspended in the hydrogel.

7. The method according to claim 1, wherein the nanostructures are sized and adapted for endocytosis by tissue at the target site such that the therapeutic agent is released within cells of the tissue when inductively heated.

8. The method according to claim 1, wherein the treatment agent includes a drug and/or a drug adjuvant or sensitizer that is insoluble or sparingly soluble.

9. The method according to claim 1, wherein the nanostructures are conductive carbon nanotubes that effectively encapsulate and isolate the composition from interaction with surrounding tissue prior to inductive heating so that the composition is released substantially only when inductively heated.

10. A method for delivering therapeutic agent to a target tissue at a tissue site, the method comprising the steps of
loading the therapeutic agent into interior spaces of conductive nanoparticles such that the nanoparticles shield the agent from interaction with surrounding tissue,
wherein the nanoparticles are coated or functionalized to constitute a delivery vehicle that selectively attaches to or is incorporated in the target tissue, and
applying an electromagnetic field to inductively heat the conductive nanoparticles when they are at the target tissue site to release the therapeutic agent at or within cells of the target tissue thereby selectively treating the target tissue.

11. The method according to claim 10, wherein the therapeutic agent is insoluble or sparingly soluble in aqueous media, and/or wherein the therapeutic agent includes a drug and/or a sensitizer which is at least one of highly toxic, sparingly soluble, or otherwise problematic to deliver to a target tissue in vivo.

12. The method according to claim 2, wherein the hydrogel is a temperature sensitive gel, and, prior to loading the method further comprises combining the therapeutic agent and the temperature sensitive gel at a temperature above the gel-sol transition temperature of the temperature sensitive gel, wherein the gel is a fluid.

13. The method according to claim 1, wherein the nanostructure is at least one of a nanotube, a nanocone, a nanohorn, a nanoporous structure, and a nanocage; and/or wherein the nanostructure is made from a material of at least one of a carbon, a gold, a silver, a platinum, a silicon, an iron, a cobalt, an iron-platinum, a iron-cobalt, a conductive polymer, and a metal ferrite.

14. The method according to claim 12, wherein the temperature sensitive gel includes at least one of an aqueous solvent and a non-aqueous solvent, and wherein the temperature sensitive aqueous gel comprises at least one of a gelatin, a starch, an agar, an agarose, a poly(ethylene oxide) (PEO), a poly(N-isoproprylacrylamide) (pNIPAAm), and a poly(propylene oxide) (PPO).

15. The method according to claim 14, wherein the non-aqueous solvent is at least one of an ethanol, a methanol, an ether or a solvent or precipitant for the therapeutic agent or a component thereof.

16. The method according to claim 1, wherein the gel has a transition temperature between about 32° C. and 45° C., or for in vivo use wherein the gel is compounded of one or more components or precursors to have a gel-sol transition temperature or other phase transition at a temperature above body temperature.

17. A nanostructure treatment construct for targeted intracellular drug delivery and temporally regulated release of an agent, the construct comprising:
a nanostructure, having open ends and a hollow interior or nanotube configuration; and,
a composition, comprising a therapeutic agent and a gel, located in the hollow interior of the nanostructure,
wherein the nanostructure shields the therapeutic agent from interaction with its surrounding until a triggered on-command release from the nanostructure.

18. The construct according to claim 17, wherein the gel is characterized by a temperature-induced phase-change transition, such that the composition is releasable from the nanostructure for delivery by inductive heating.

19. The construct according to claim 17, wherein the hollow nanostructure further comprises an exterior surface that is chemically functionalized for at least one function selected from: solubility, drug absorption, responsivity to pH, responsivity to enzyme catalysis, and responsivity to ambient biological or fluid environment or temperature.

20. The construct according to claim 17, wherein the exterior nanostructure surface is treated with nitric acid, wherein functionalized cellular uptake of a resulting nitric acid-treated nanostructure is greater than that of a control hollow nanostructure not so treated and otherwise identical.

21. The construct according to claim 20, wherein the exterior nanostructure surface is non-covalently functionalized with phospholipid-polyethylene glycol (PL-PEG) wherein a resulting PEG-functionalized nanostructure is more hydrophilic and displays prolonged circulation in the bloodstream in comparison to a control nanostructure not so functionalized.

22. The construct according to claim 17, wherein the hollow nanotube is a PEG-functionalized carbon nanotube about 50 nm in diameter and about 200-1000 nm in length.

23. The construct according to claim 17, wherein the nanostructure is functionalized to further comprise amine-terminated polyethylene glycol phospholipids (PL-PEG-NH₂) and has a plurality of amine terminals for further conjugation of biomolecular adducts.

24. The construct according to claim 17, wherein the hollow nanostructure is at least one of a nanotube, a nanocone, a nanohorn, a nanoporous structure, and a nanocage, and is made from at least one material selected from a carbon, a gold, a silver, a platinum, a silicon, an iron, a cobalt, an iron-platinum, an iron-cobalt, a conductive polymer, and a metal ferrite.

25. The construct according to claim 18, wherein the temperature sensitive gel is or includes at least one of a gelatin, a starch, an agar, an agarose, a poly(ethylene oxide) (PEO), a poly(N-isoproprylacrylamide) (pNIPAAm), a poly(propylene oxide) (PPO), a poly(N,n-diethylacrylamide), a poly(N-isopropylmethacrylamide), a poly(N-cyclopropylacrylamide), an hydroxypropyl cellulose, a methyl cellulose, an hydroxypropylmethyl cellulose and an ethylhydroxyethyl cellulose.

26. The construct according to claim 17, wherein the nanostructure has a non-covalently functionalized surface to improve compatibility with an intended medium, formulation or target.
